(19) United States
(12) Patent Application Publication
(10) Pub. No.: US 2022/0365017 A1
(43) Pub. Date: Nov. 17, 2022

(54) PROGRAMMABLE MULTIFUNCTION PH MEASUREMENT SYSTEM BASED ON MOBILE COMMUNICATION

(71) Applicant: National Pingtung University of Science and Technology, Neipu Township (TW)

(72) Inventors: Chang-Hsien Tai, Pingtung County (TW); Yao-Nan Wang, Neipu Township (TW); Cheng-Huei Yang, Neipu Township (TW); Chin-Lung Chang, Neipu Township (TW); Tsung-Che Wu, Neipu Township (TW)

(21) Appl. No.: 17/646,121

(22) Filed: Dec. 27, 2021

(30) Foreign Application Priority Data
May 13, 2021 (TW) 110117287

(51) Int. Cl. G01N 27/12; H04M 1/02

(52) U.S. Cl. CPC G01N 27/12; H04M 1/0266; H04M 2250/12; H04M 2250/10

(57) ABSTRACT

A pH measurement system includes a measurement apparatus that is capable of connection to a mobile device and a server and that includes a measuring instrument and a monitoring device. The measuring instrument includes a tube, and a sensor rod that is disposed is the tube and that is for sensing a pH value of a measurement target. The monitoring device includes a microcontroller communicatively connected to the sensor rod, an operation module for operation by a user, a communication module for communication with the mobile device and the server, and a display module for displaying information. The microcontroller provides the pH value to the mobile device or the server through the communication module.

### CROSS-REFERENCE TO RELATED APPLICATION

This application claims priority of Taiwanese Invention Patent Application No. 110117287, filed on May 13, 2021.

### FIELD

The disclosure relates to a system for measuring a physical quantity, and more particularly to a programmable multifunction pH measurement system based on mobile communication.

### BACKGROUND

In chemistry, pH is a scale that indicates a concentration of hydrogen ions H⁺, and is the most common measurement standard for presenting acidity and alkalinity of a solution. A variety of pH measurement apparatuses have been developed to meet the needs of various fields.

Because different fields may have different needs in terms of pH measurement, differently designed conventional pH measurement apparatuses that are tailored toward the different needs, such as one-time measurement or continuous measurement, fixed-position measurement or handheld measurement, for data control, etc., are now available. However, no single apparatus has sufficient versatility to accommodate different needs of the pH measurement.

In addition, with the rapid advancement of technology in today's generation, measurement apparatuses that only provide measurement data alone can no longer meet the practical needs in data management, interpretation and analysis.

### SUMMARY

Therefore, an object of the disclosure is to provide a programmable multifunction pH measurement system that has high versatility and that can achieve efficient data management, interpretation and analysis via mobile communication.

According to the disclosure, the programmable multifunction pH measurement system based on mobile communication includes a measurement apparatus. The measurement apparatus includes a measuring instrument and a monitoring device. The measuring instrument includes a tube that defines an installation space, and a sensor rod that is disposed in the installation space and that is configured to sense a pH value of a measurement target. The monitoring device is detachably mounted to the measuring instrument, and includes a microcontroller that is communicatively connected to the sensor rod, an operation module that is electrically connected to the microcontroller and that is configured for operation by a user, a communication module that is electrically connected to the microcontroller, and a display module that is electrically connected to the microcontroller for displaying information provided by the microcontroller and related to a measurement of the pH value. The microcontroller is configured to receive the pH value from the sensor rod, is to be communicatively connected to mobile device through the communication module for providing the pH value to the mobile device based on a user operation performed on the operation module, and is to be communicatively connected to a server through the communication module for providing the pH value to the server based on another user operation performed on the operation module.

### BRIEF DESCRIPTION OF THE DRAWINGS

Other features and advantages of the disclosure will become apparent in the following detailed description of the embodiment (s) with reference to the accompanying drawings, of which:

FIG. 1 is a schematic diagram illustrating a first embodiment of a programmable multifunction pH measurement system based on mobile communication according to this disclosure;

FIG. 2 is a schematic diagram illustrating a measurement apparatus of the first embodiment;

FIG. 3 is a block diagram illustrating a monitoring device of the first embodiment;

FIG. 4 is a schematic diagram illustrating that a mobile device graphically shows measurement data acquired using the first embodiment; and

FIG. 5 is a schematic diagram illustrating a tube of a second embodiment of a programmable multifunction pH measurement system based on mobile communication according to this disclosure.

### DETAILED DESCRIPTION

Before the disclosure s described in greater detail, it should be noted that where considered appropriate, reference numerals or terminal portions of reference numerals have been repeated among the figures to indicate corresponding or analogous elements, which may optionally have similar characteristics.

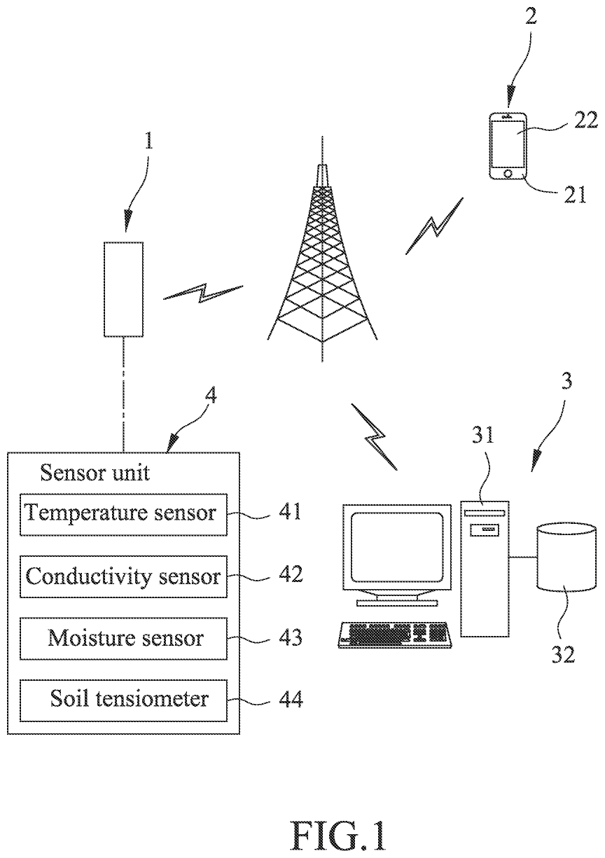

Referring to FIG. 1, a first embodiment of a programmable multifunction pH measurement system based on mobile communication according to this disclosure is shown to include a measurement apparatus 1, a mobile device 2 that is communicatively connected to the measurement apparatus 1, a server 3 that is communicatively connected to the measurement apparatus 1, and a sensor unit 4 that is connected to the measurement apparatus 1. The communicative connections between the measurement apparatus 1 and the mobile device 2 and between the measurement apparatus 1 and server 3 may be realized using a wireless network, such as Wi-Fi™, Bluetooth®, cellular network, etc. In some embodiments, a dedicated base station may be used to ensure stable wireless connections, but this disclosure is not limited in this respect.

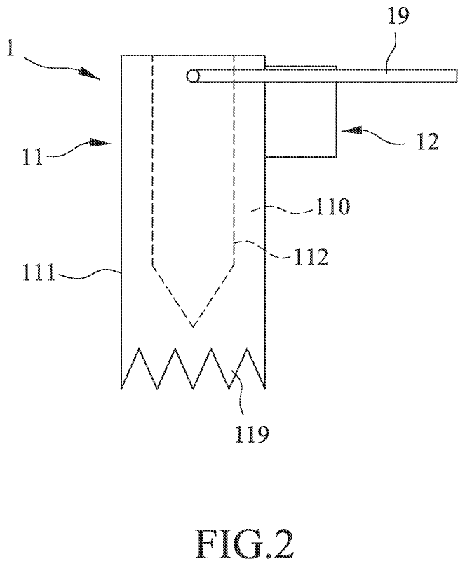

Further referring to FIG. 2, the measurement apparatus 1 includes a measuring instrument 11, monitoring device 12 that is detachably mounted to the measuring instrument 11, and a handle 19 that is connected to the measuring instrument The measuring instrument 11 includes a tube 111 that defines an installation space 110, and a sensor rod 112 that is disposed in the installation space 110 and that is configured to sense a pH value of a measurement target (e.g., soil). The handle 19 is rotatable relative to the tube 111 about an axis that is transverse or perpendicular to both of a lengthwise direction of the tube 111 and a lengthwise direction of the handle 19. For example, a shaft may used to serve the axis and to rotatably interconnect the tube 111 and the handle 19. The tube 111 may be made of, for example, metal, wood, plastic materials, other suitable materials, or any combination thereof, and has a saw-toothed end portion 119 for positioning the measuring instrument 11. The saw-toothed end portion 119 may be useful for fixed-position measurement. For example, a user may use the saw-toothed end portion 119 to insert the tube 111 into the soil, so as to fix the measuring instrument 11 in place for measurement. In this embodiment, the sensor rod 112 includes an electrode assembly (not shown) adapted for measuring an electrical conductivity of the measurement target, which can be converted into a hydrogen ion concentration of the measurement target, so as to obtain the pH value of the measurement target.

The handle 19 is rotatable between a storage position and a use position. In the storage position, the handle 19 is parallel to the tube 111. In the use position, the handle 19 is transverse or perpendicular to the tube 111, and a user can hold the handle 19 to perform handheld measurement, attach the handle 19 to other objects for positioning the measuring instrument 11, so as to perform long-term measurement. When the handle 19 is in the storage position, space required for accommodating the measuring instrument 11 may be reduced, thereby facilitating storage or carrying.

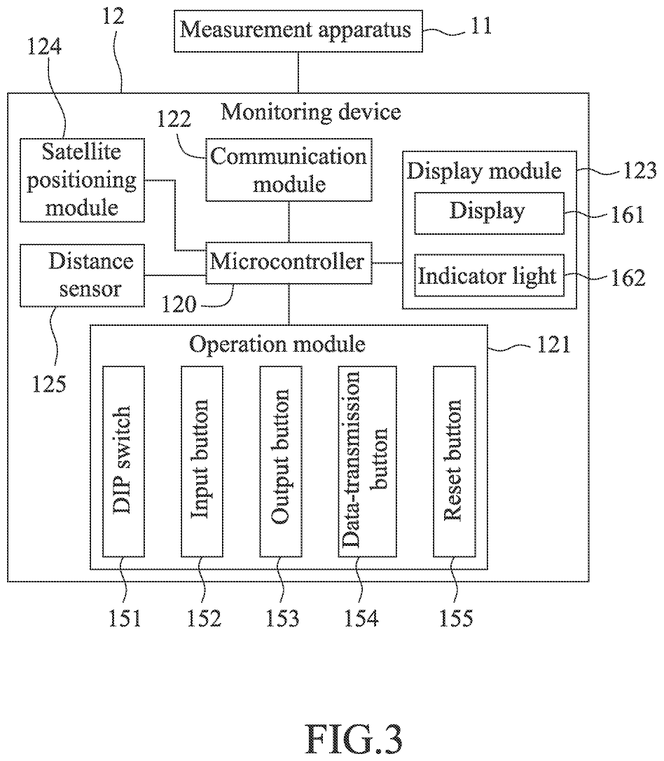

Referring to FIGS. 2 and 3, the monitoring device 12 includes a microcontroller 120 that is communicatively connected to the sensor rod 112, an operation module 121 that is electrically connected to the microcontroller 120 and that is configured for operation by a user, a communication module 122 that is electrically connected to the microcontroller 120, a display module 123 that is electrically connected to the microcontroller 120 for displaying information provided by the microcontroller 120 and related to a measurement of the pH value, a satellite positioning module 124 (e.g., the global positioning system, GPS) that is electrically connected to the microcontroller 120 and that is configured to generate location information of the monitoring device 12, and a distance sensor 125 that is electrically connected to the microcontroller 120 and that uses, for example but not limited to, ultrasonic sound waves to sense depth in the measurement target at which the measuring instrument 11 measures the pH value (referred to as measurement depth) and generate depth information that indicates the measurement depth. In this embodiment, the display module 123 includes a display 161 for displaying the information provided by the microcontroller 120 and related to a measurement of the pH value, and an indicator light 162 disposed to emit light upon receipt of a warning signal from the microcontroller 120.

The operation module 121 includes a DIP switch 151, an input button 152, an output button 153, a data-transmission button 154 and a reset button 155.

The DIP switch 151 is operable by the user to input one or more settings for the microcontroller 120, which is a programmable arithmetic and control unit in this embodiment. In some embodiments, the DIP switch 151 is used to set warning ranges for parameters (e.g., the measurement depth) related to the measurement of the pH value. When a parameter falls within the corresponding warning range, the microcontroller 120 may send the warning signal to the indicator light 162, so as to control the indicator light 162 to emit light. In this embodiment, the indicator light 162 includes a light emitting diode (LED) that emits green light (referred to as greed LED), and an LED that emits red light (referred to as red LED), where the greed LED and the red LED respectively correspond to a normal state (e.g., none of the parameters falls within the corresponding warning range) and a warning state (e.g., a parameter falls within the corresponding warning range).

The input button 152 is operable by a user to confirm input of the setting(s) (e.g., settings of the warning ranges) into the microcontroller 120. The output button 153 is operable by a user to make the microcontroller 120 control the display module 123 to display information related to the measurement or the pH value. The data-transmission button 154 is operable by a user to make the microcontroller 120 transmit data related to the pH value to the mobile device 2 and/or the server 3. The reset button 155 is operable by a user to reset the programmable multifunction pH measurement system to an initial state, so as to instantly resolve problems that may result from complex manual settings.

The satellite positioning module 124 is used to generate the location information of a measurement spot where the measurement is performed, and the distance sensor 125 is used to generate the depth information that advises the user of the measurement depth (e.g., a measurement depth in water, a measurement depth in soil, a measurement depth in food, etc.). The use of the distance sensor 125 can ensure the same measurement depth in different measurement environments, so as to facilitate analysis on data thus measured (e.g., pH values, referred to as measurement data hereinafter). Taking soil tests as an example, using the distance sensor 125 can make sure that the measurements are all performed at the same measurement depth in soil, so the precision of the subsequent analysis on the measurement data may be optimized.

Referring to FIGS. 1 and 3, in this embodiment, the mobile device 2 may be a smartphone, but this disclosure is not limited in this respect. The mobile device 2 is communicatively connected to the microcontroller 120 through the communication module 122, and includes a device body 21 and an operation interface 22. In practice, the operation interface 22 may be a touch screen, but this disclosure is not limited in this respect. In addition to operating the data-transmission button 154 to make the monitoring device 12 transmit the data related to the pH value to the mobile device 2, the user can also use the mobile device 2 to control the monitoring device 12 to periodically transmit the data to the mobile device 2 by operating the operation interface 22, which is suitable for the scenario of continuous measurement. Furthermore, the user can also operate the operation interface 22 to input the settings for the monitoring device 12, to control operation of the monitoring device 12, to monitor the measurement performed by the measuring instrument 11, and to query the measurement data

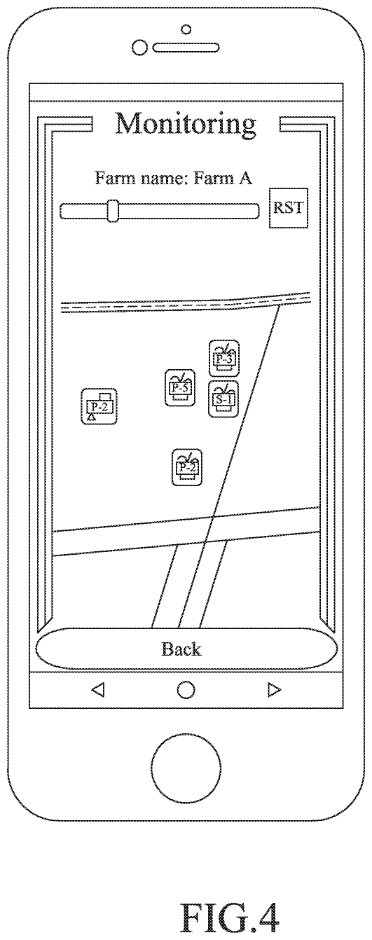

The mobile device 2 receives the measurement data (e.g., the pH values measured by the measurement apparatus 1) and the location information that is generated by the satellite positioning module 124 and that indicates the locations where the measurements were performed, and controls the display module 123 to graphically show the measurement data on an electronic map, as illustrated in FIG. 4. In practice, the mobile device 2 may be installed with an application that has a plurality of predefined ranges set for the pH value, and a plurality of different colors set to respectively correspond to the predefined ranges. The application is configured to mark a data point for each measured pH value on an electronic map at a position corresponding to the measurement spot where the measurement of the pH value was performed, and the data point is marked on the electronic map using one of the different colors that corresponds to one of the predefined ranges in which the pH value falls. For example, the predefined ranges for the pH value may include a range of between 5.1 and 6.0 that corresponds to a red color, a range of between 6.1 and 7.0 that corresponds to a green color, and a range of between 7.1 and 8.0 that corresponds to a blue color. As a result, each piece of the measurement data may be shown as a data point on the electronic map at a position corresponding to the measurement spot, with a color corresponding to the pH value as indicated by the piece of the measurement data. Such graphical presentation of information has a better readability for a user, thereby facilitating the user to quickly become aware of characteristics of the measurement spots in terms of acidity and alkalinity (e.g., being homogeneous or heterogeneous), and promoting efficiency in making decisions for following actions to be taken with respect to the measurement target. Taking the soil preparation that is performed before growing crops as an example, after an initial soil preparation, the system of the embodiment can be used to measure the pH values of the soil at different measurement spots. The measurement data will be shown on the electronic map in the form of data points with colors that correspond to the pH values measured at the measurement spots, so the user can immediately become aware of the condition of the soil in terms of acidity and alkalinity, and can thus quickly make decisions on the following actions to be taken with respect to the soil. When the data points on the electronic map have the same color, the soil is homogeneous in terms of acidity and alkalinity, no further improvement is required for the soil. When the data points on the electronic map have different colors, the soil is heterogeneous in terms of acidity and alkalinity, and further improvement for the soil is required, so as to ensure that the crops can be grown in the same environment. Therefore, the colored data points on the electronic map can assist the user in quickly evaluating the condition of the soil, and reducing time required for determining the following actions to be taken with respect to the soil.

In addition, the application is configured in such as way that, when the mobile device 2 displays the data points on the electronic map, the user may directly select (e.g., by tapping on the touch screen) one or more of the data points to make the mobile device 2 show the measurement data corresponding to the data point(s), so the user can easily query the measurement data, can easily check multiple pieces of the measurement data together instead of checking the pieces of the measurement data individually, and can thus easily make a conclusion based on the pieces of the measurement data that are simultaneously shown on the electronic map. Because of the portability of the mobile device 2, it is convenient for the user to perform required operation (e.g., input settings for the microcontroller 120, querying the measurement data, etc.), and instant evaluation on the measurement data can be achieved. Furthermore, the mobile device 2 can be operated to configure a proper interface for data output in advance, thereby promoting efficiency for the entire measurement process.

The server 3 is communicatively connected to the microcontroller 120 through the communication module 122 for receiving the measurement data, and includes a host computer 31, and a database 32 that is electrically connected to the host computer 31. Although the server 3 is not portable like the mobile device 2, the server 3 may perform the same functions as the mobile device 2, such as inputting settings for microcontroller 120, controlling the measurement apparatus 11, monitoring the measurement, querying the measurement data, etc. Furthermore, when cooperating with the database 32 that may have historic data, a data management system and an interactive interface pre-established therein, the ability to perform relatively high-speed processing and handle a relatively large amount of data allows the host computer 31 to perform data integration and/or subsequent processing, so as to present the measurement data and the analysis result in a specific manner, thus promoting efficiency of data management and evaluation.

In this embodiment, the sensor unit 4 includes a temperature sensor 41 disposed to sense temperature of the measurement target, a conductivity sensor 42 disposed to sense electrical conductivity of the measurement target, a moisture sensor 43 disposed to sense moisture of the measurement target (e.g., soil moisture content), and a soil tensiometer 44 disposed to sense soil moisture tension of the measurement target (which can be converted into soil compactness) when the measuring target is soil. By virtue of the measurements performed by the sensor unit 4 in cooperation with the measurement of the pH value, a more comprehensive environmental inspection can be performed.

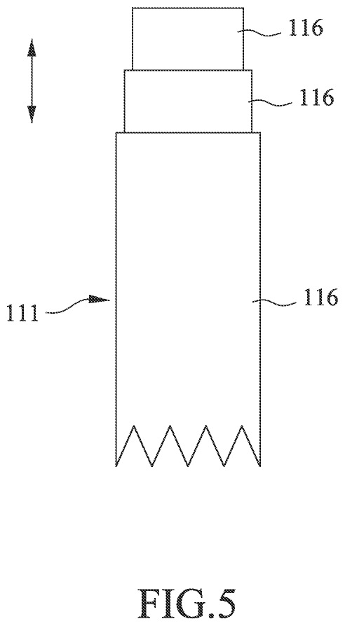

This disclosure further provides a second embodiment of the programmable multifunction pH measurement system based on mobile communication, which is similar to the first embodiment. Referring to FIG. 5, the second embodiment differs from the first embodiment in that the tube 111 of the second embodiment is a telescopic tube that includes a plurality of tube segments 116 that are sleeved one on another and that are movable relative to each other. By virtue of the tube segments 116, a length or the tube 111 is adjustable for different measurement depths. In this embodiment, when the measurement apparatus 11 has wires (not shown) disposed in the tube 111 for making connection among the components of the measurement apparatus 11, a reel (not shown) may be used to adjust the lengths of the wires to fit the length of the tube 111, so as to ensure stable electric and/or communicative connections among the components of the measurement apparatus 11.

To sum up, in the embodiments of the programmable multifunction pH measurement system based on mobile communication according to this disclosure, the monitoring device 12 that is communicatively connected to the mobile device 2 and the server 3 enables the user to input the settings for the microcontroller 120 of the monitoring device 12, to control the operation of the monitoring device 12, to monitor the measurement performed by the measurement apparatus 11, and to query the measurement data through the mobile device 2 or the server 3 that is communicatively connected to the monitoring device 12, so as to facilitate instant integration of a large amount of the measurement data to generate graphical information for enhanced readability, and the user can thus easily derive conclusion from the measurement data. In addition, the handle 19 that is rotatable relative to the tube 111 promotes the versatility of the measurement apparatus 11 for different needs of measurements.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass ail such modifications and equivalent arrangements.

What is claimed is:

1. A programmable multifunction pH measurement system based on mobile communication, comprising:
a measurement apparatus that includes
a measuring instrument including a tube that defines an installation space, and a sensor rod that is disposed in the installation space and that is configured to sense a pH value of a measurement target; and
a monitoring device detachably mounted to said measuring instrument, and including a microcontroller that is communicatively connected to said sensor rod, an operation module that is electrically connected to said microcontroller and that is configured for operation by a user, a communication module that is electrically connected to said microcontroller, and a display module that is electrically connected to said microcontroller for displaying information provided by said microcontroller and related to a measurement of the pH value;
wherein said microcontroller is configured to receive the pH value from said sensor rod, to be communicatively connected to a mobile device through said communication module for providing the pH value to said mobile device based on a user operation performed on said operation module, and to be communicatively connected to a server through said communication module for providing the pH value to said server based on another user operation performed on said operation module.

2. The programmable multifunction pH measurement system of claim 1, wherein said measurement apparatus further includes a handle that is connected to said tube, and that is rotatable relative to said tube about an axis transverse to a lengthwise direction of said tube.

3. The programmable multifunction pH measurement system of claim 1, wherein said tube has a saw-toothed end portion for positioning said measuring instrument.

4. The programmable multifunction pH measurement system of claim 1, wherein said operation module includes a DIP switch operable by the user to input a setting for said microcontroller, an input button operable by the user to confirm input of the setting into said microcontroller, an output button operable by the user to make said microcontroller control said display module to display information related to the measurement of the pH value, and a data-transmission button operable by the user to make said microcontroller transmit data related to the pH value to one of the mobile device and the server.

5. The programmable multifunction pH measurement system of claim 4, wherein said operation module further includes a reset button operable by the user to reset said pH measurement system to an initial state.

6. The programmable multifunction pH measurement system of claim 1, wherein said display module includes a display for displaying the information provided by said microcontroller and related to a measurement of the pH value, and an indicator light disposed to emit light upon receipt of a warning signal from said microcontroller.

7. The programmable multifunction pH measurement system of claim 1, further comprising the mobile device, the mobile device being a smartphone.

8. The programmable multifunction pH measurement system of claim 1, further comprising the mobile device, wherein said mobile device is installed with an application that configured to mark a data point representing the pH value on an electronic map at a position which corresponds to a measurement spot where the measurement of the pH value was performed;
wherein the application is set with a plurality of predefined ranges for the pH value, and a plurality of different colors that respectively correspond to the predefined ranges;
wherein the data point is marked on the electronic map using one of the different colors that corresponds to one of the predefined ranges in which the pH value falls.

9. The programmable multifunction pH measurement system of claim 1, wherein said monitoring device further includes a satellite positioning module that is electrically connected to said microcontroller and that is configured to generate location information of said monitoring device.

10. The programmable multifunction pH measurement system of claim 1, wherein said monitoring device further includes a distance sensor that is electrically connected to said microcontroller and that is configured to generate depth information which indicates a depth in the measurement target at which said measuring instrument measures the pH value.

11. The programmable multifunction pH measurement system of claim 1, wherein said tube is a telescopic tube that includes a plurality of tube segments which are movable relative to each other.

12. The programmable multifunction pH measurement system of claim 1, further comprising a sensor unit connected to said measurement apparatus, wherein said sensor unit includes a temperature sensor disposed to sense temperature of the measurement target, a conductivity sensor disposed to sense electrical conductivity of the measurement target, a moisture sensor disposed to sense moisture of the measurement target, and a soil tensiometer disposed to sense soil moisture tension of the measurement target when the measuring target is soil.
